(19) United States
(12) Patent Application Publication
(10) Pub. No.: US 2020/0016305 A1
(43) Pub. Date: Jan. 16, 2020

(54) Device for Stimulating Milk Flow

(71) Applicant: Eran Weinberg, Los Angeles, CA (US)

(72) Inventor: Eran Weinberg, Los Angeles, CA (US)

(21) Appl. No.: 16/507,332

(22) Filed: Jul. 10, 2019

Related U.S. Application Data
(60) Provisional application No. 62/696,205, filed on Jul. 10, 2018.

(51) Int. Cl. A61M 1/06

(52) U.S. Cl. CPC A61M 1/062; A61B 5/4288

(57) ABSTRACT

A device for stimulating milk flow, comprising: a housing; a vibrating component; a heating component; a power switch; a heating button; wherein said housing comprises a top portion and a bottom portion; wherein said top portion comprises said power switch and said heating button; wherein said housing houses said vibrating component and said heating component; and wherein said bottom portion comprises an engagement surface.

This application is a Non-Provisional patent application that claims the benefit of and priority to U.S. Provisional Patent Application No. 62/696,205, filed on Jul. 10, 2018, titled Device for Stimulating Milk Flow, by inventor Eran Weinberg, the contents of which are expressly incorporated herein by this reference.

### FIELD OF USE

The present disclosure generally relates to the field of improving milk flow of breastfeeding mothers. More specifically, the present disclosure generally relates to a device that may be used or worn to improve the flow of milk during breastfeeding.

### BACKGROUND

Many women who breastfeed their babies experience difficulty with flow of milk. There are a number of issues that a nursing mother may experience that may make breastfeeding difficult or uncomfortable.

Generally, a human mother's mammary glands begin to generate and store limited quantities of milk after the birth of a child. Once generated and stored in the mammary glands, the milk then travels through the milk duct to the nipple when a suction is applied.

If a mother is not able to properly move the milk out, the mother may experience engorgement or have clogged ducts, wherein the flow of milk is blocked such that the milk does not have an effective egress point. This clogging may also lead to other complications such as mastitis, or infection in the breast. Alternatively, a mother may simply experience a slow flow or difficulty fully emptying the mammary glands.

In these cases, improved milk flow and effectively emptying of the breast may provide relief to the mother and allow the breastfeeding to continue without issue.

Thus, what is needed is a device to assist breastfeeding women in improving the flow of breastmilk while nursing or pumping.

### SUMMARY

The present specification discloses a device for improving milk flow of lactating mothers while nursing or pumping. The device may preferably comprise both a vibrating component and a heating component that are used simultaneously, consecutively, and/or separately as determined by the user. The device may be concave to matingly cradle the user's breast when in use.

One embodiment may be a device for stimulating milk flow, comprising: a housing; a vibrating component; a heating component; a power source; and one or more controllers; wherein the housing may house the vibrating component and the heating component; and wherein the one or more controllers may be configured to control the vibrating component and the heating component, such that both vibration and heat may be provided to a lactating user's breast simultaneously, which stimulates milk flow. The device for stimulating milk flow may further comprise a heating safety component; wherein the heating safety component may comprise a thermostat. The heating safety component may be configured to deactivate the heating component upon detection of a pre-determined temperature level; and the pre-determined temperature level may be a temperature level lower than a temperature level that would cause injury to the user of the device for stimulating milk flow after prolonged contact with the user's skin. The injury may be at least a third-degree burn. The heating safety component may cause the heating component to output varying levels of heat, such that the temperature generated increases and decreases in a pre-determined manner in a single use session. At least one of the one or more controllers may be a heat selection switch. The heat selection switch may allow selection of at least high heat, low heat, or no heat. A bottom portion of the housing may comprise an engagement surface. The engagement surface may comprise silicone. The engagement surface may be contoured. The engagement surface may be concave. A top portion of the housing may comprise the one or more controllers. At least one of the one or more controllers may be a heating button. The heating button may be located on a top portion of the housing. The device for stimulating milk flow may further comprise a charging port.

Another embodiment may be a system for stimulating milk flow, comprising: two vibrating and heating devices; wherein the each of the two vibrating and heating devices may comprise a housing, a vibrating component, a heating component, a heating safety component, an engagement surface, one or more controllers, a power source, and a charging port; wherein each of the two housings contains one of the two vibrating components and one of the two heating components; and wherein each of the one or more controllers may be configured to control the vibrating component and the heating component, such that both vibration and heat may be provided to a lactating user's breast simultaneously in each of the two vibrating and heating devices. The two vibrating and heating devices may be physically connected to one another when charging.

Another embodiment may be a device for stimulating milk flow, comprising: a housing; a vibrating component; a heating component; a power source; a charging port; a heating safety component; and one or more controllers; wherein the housing may house the vibrating component and the heating component; wherein the one or more controllers may be configured to control the vibrating component and the heating component, such that both vibration and heat may be provided to a lactating user's breast simultaneously, which stimulates milk flow; wherein the heating safety component may comprise a thermostat; wherein the heating safety component may be configured to deactivate the heating component upon detection of a pre-determined temperature level; wherein the pre-determined temperature level may be a temperature level lower than a temperature level that would cause injury to the user of the device for stimulating milk flow after prolonged contact with the user's skin; wherein the injury may be at least a third-degree burn; wherein the heating safety component may cause the heating component to output varying levels of heat, such that the temperature generated increases and decreases in a pre-determined manner in a single use session; wherein at least one of the one or more controllers may be a heat selection switch; wherein the heat selection switch allows selection of at least high heat, low heat, or no heat; wherein a bottom portion of the housing may comprise an engagement surface; wherein the engagement surface substantially may comprise silicone; wherein the engagement surface may be contoured; wherein the engagement surface may be concave; wherein a top portion of the housing may comprise the one or more controllers; wherein the at least one of the one or more controllers may be a heating button; and wherein the heating button may be located on a top portion of the housing.

### BRIEF DESCRIPTION OF THE ILLUSTRATIVE EMBODIMENTS

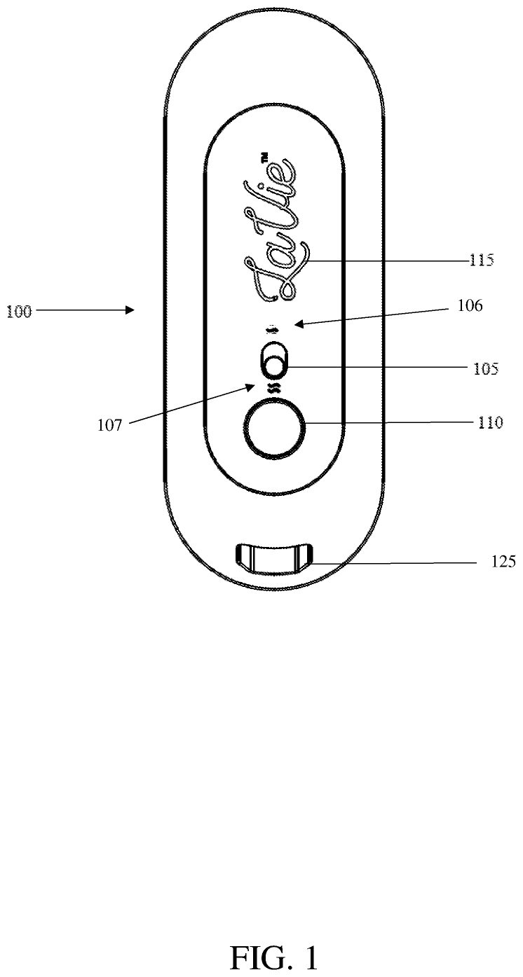

FIG. 1 illustrates a top view of one embodiment of the device.

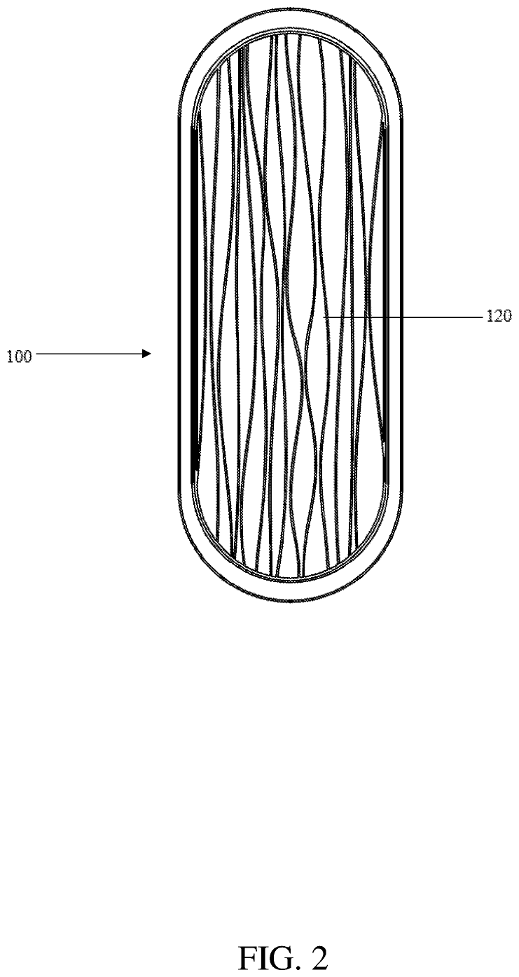

FIG. 2 illustrates a bottom view of one embodiment of the device.

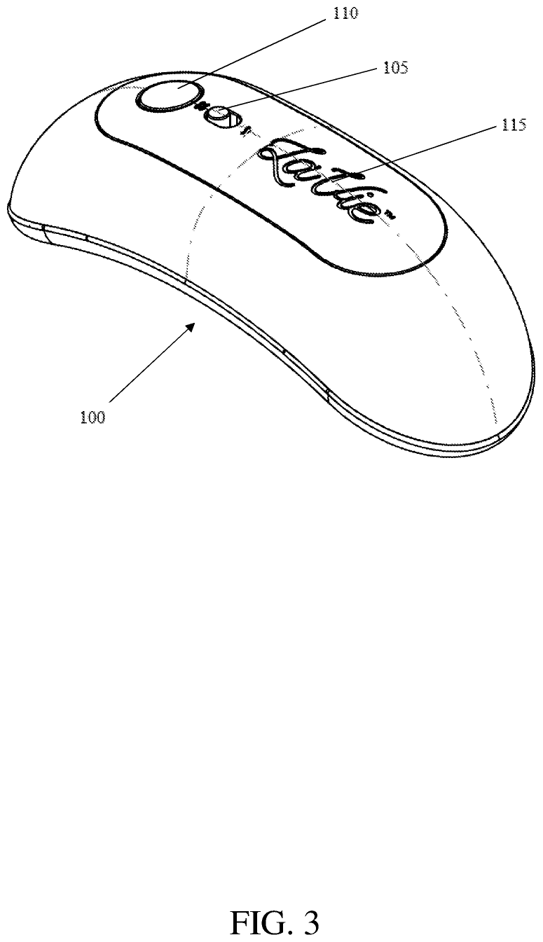

FIG. 3 illustrates a top perspective view of one embodiment of the device.

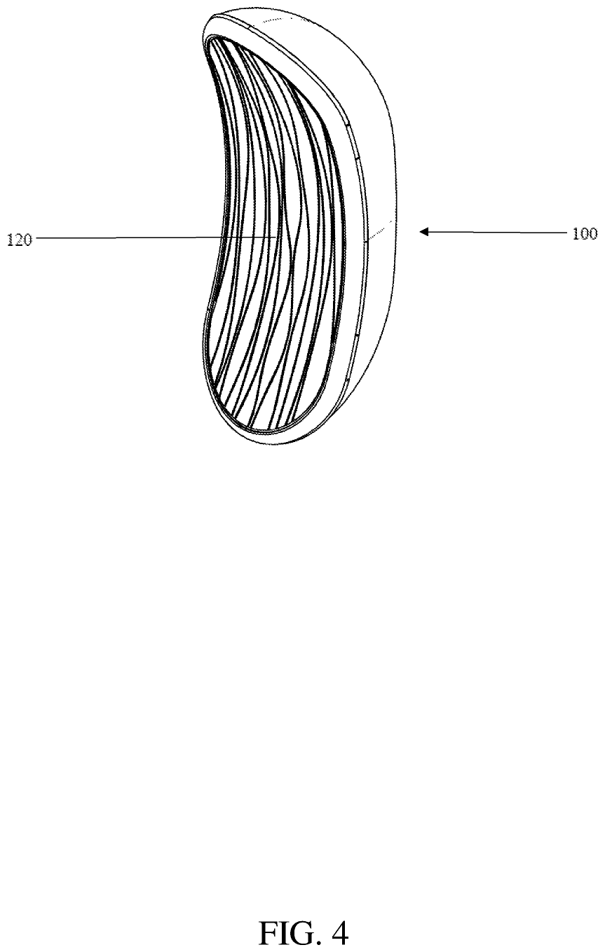

FIG. 4 illustrates a bottom perspective view of one embodiment of the device.

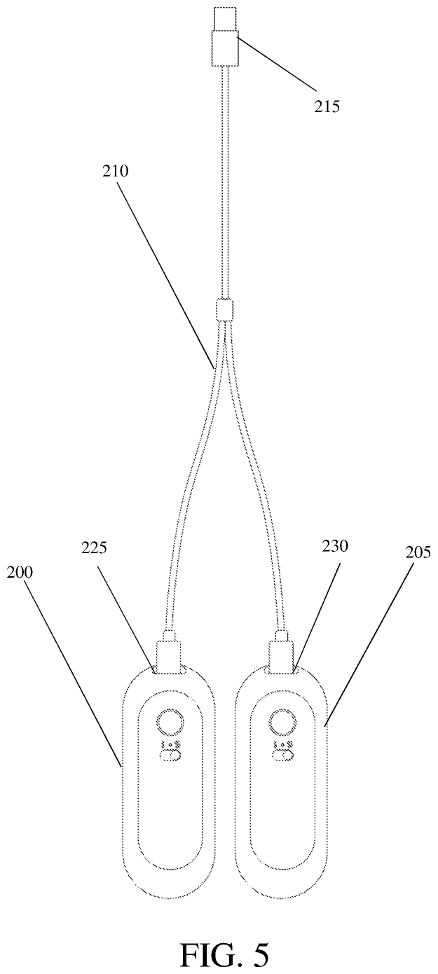

FIG. 5 illustrates a top view of one embodiment of two devices with charging cables.

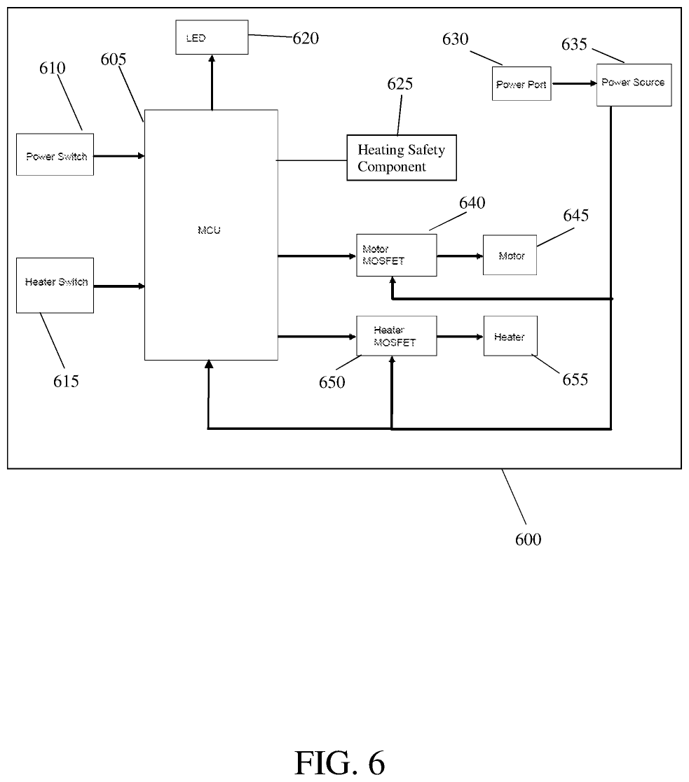

FIG. 6 illustrates a block diagram of the components of the device.

### DETAILED DESCRIPTION OF THE ILLUSTRATIVE EMBODIMENTS

In the following detailed description of various embodiments, numerous specific details are set forth in order to provide a thorough understanding of various aspects of the embodiments. However, these embodiments may be practiced without some or all of these specific details. In other instances, well-known methods, procedures, and/or components have not been described in detail so as not to unnecessarily obscure aspects of the embodiments.

While multiple embodiments are disclosed, still other will become apparent to those skilled in the art from the following detailed description. As will be realized, these embodiments are capable of modifications in various obvious aspects, all without departing from the spirit and scope of protection. Accordingly, the graphs, figures, and the detailed descriptions thereof, are to be regarded as illustrative in nature and not restrictive. Also, the reference or non-reference to a particular embodiment shall not be interpreted to limit the scope of protection.

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are signify both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that may be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all embodiments of this application including, but not limited to, steps in disclosed methods. Thus, if there are a variety of additional steps that may be performed it is understood that each of these additional steps may be performed with any specific embodiment or combination of embodiments of the disclosed methods.

The present methods and systems may be understood more readily by reference to the following detailed description of preferred embodiments and the examples included therein and to the Figures and their previous and following description.

In the following description, certain terminology is used to describe certain features of one or more embodiments. For purposes of the specification, unless otherwise specified, the term “substantially” refers to the complete or nearly complete extent or degree of an action, characteristic, property, state, structure, item, or result. For example, in one embodiment, an object that is “substantially” located within a housing would mean that the object is either completely within a housing or nearly completely within a housing. The exact allowable degree of deviation from absolute completeness may in some cases depend on the specific context. However, generally speaking, the nearness of completion will be so as to have the same overall result as if absolute and total completion were obtained. The use of “substantially” is also equally applicable when used in a negative connotation to refer to the complete or near complete lack of an action, characteristic, property, state, structure, item, or result.

As used herein, the terms “approximately” and “about” generally refer to a deviance of within 5% of the indicated number or range of numbers. In one embodiment, the term “approximately” and “about”, may refer to a deviance of between 0.001-10% from the indicated number or range of numbers.

One embodiment may be a device for stimulating milk flow, comprising: a housing; a vibrating component; a heating component; a power switch; a heating button; wherein the housing may comprise a top portion and a bottom portion; wherein the top portion may comprise the power switch and the heating button; wherein the housing may house the vibrating component and the heating component; and wherein the bottom portion may comprise an engagement surface.

FIG. 1 illustrates a top view of one embodiment of the device. As shown in FIG. 1, the device 100 may comprise a top portion 115. The top portion 115 may comprise a heat switch 105, a power button 110, and a power connect 125. The device 100 may further comprise a vibrating component and a heating component, which are controlled by the controllers 105, 110. The power button 110 and heat switch 105 are controllers or controller mechanisms. The power button 110 may activate the vibrating component, and, in some embodiments, allow for activation of the heating component. When the vibrating component is active, by pressing the power button 110, the heat switch 105 may be slid to activate the heating component, such that both the vibrating component and the heating component are active at the same time. The user may hold the device 100 by placing the top portion 115 in the user's hand. The vibrating component may be a motor with a weighted head to provide vibration substantially across the length of the device 100. The device 100 may comprise a rechargeable battery to power the vibrating component and heating component. The power connector 125 may be used to charge the built-in rechargeable battery. The power connector 125 may comprise a standard power connection port, such as mini usb, micro usb, usb-c, a standard power connector, or a proprietary power delivery mechanism. Alternatively, the device 100 may accept removable batteries, or receive power directly from a wall outlet or other power source. There may also be more than one rechargeable battery, such as two batteries, one for the heating component and one for the vibrating component.

In an alternative embodiment, the power button 110 may actuate different vibrating modes, such as pulse, continuous, variable, scaled variable, and off. The heat switch 105 may have one or more heating modes, such as high heat 107, low heat 106, and no heat (between 107 and 106), such that activating the heating switch changes the heating mode from one mode to another. In another alternative embodiment, the power button 110 may comprise a slidable switch, or other analog activation mechanism for selecting the vibration mode. In another embodiment, the heat switch 105 may be an analog button or a digital interface, such as a touch pad. In another embodiment, the heat and vibrating power buttons and switches may be swapped, such that the heat is first turned on and only then can the vibrating component be turned on. In another embodiment, the heat and vibrating components may be independently operated, such that the user may use one, the other, or both, as desired. In another embodiment the heating component and the vibrating component may be controlled by the same controller or controller mechanism or two different controllers. An example of a controller that can control both heat and vibration would be a digital controller.

FIG. 2 illustrates a bottom view of one embodiment of the device. As shown in FIG. 2, the device 100 may comprise an engagement surface 120. The engagement surface 120 may be configured to transmit heat and vibration generated by the heating component and vibrating component, respectively, to the user's breast to allow for stimulation and increased milk flow. The engagement surface 120 may be silicone, a hard material, a soft material, or substantially any material suitable for transmitting vibration or heat. The engagement surface 120 may also comprise a contour (such as a concave contour) such that the engagement portion 120 may cradle the user's breast to allow for an increased contact area and comfort, as compared to a substantially flat engagement surface. In an alternative embodiment, the engagement portion 120 may be substantially flat. The heating component may sit just underneath the engagement surface 120.

The device 100 may also be placed inside of a nursing bra or pumping bra, thereby being usable without the user actively holding the device 100. The edge and rounded end of the device 100 may be used to scoop and provide direct pressure on a clogged duct, thereby allowing the fluid to flow more easily and clear clogged ducts.

FIG. 3 illustrates a top perspective view of one embodiment of the device.

FIG. 4 illustrates a bottom perspective view of one embodiment of the device. As shown in FIG. 4, the engagement surface 120 may comprise a contour, in this case a substantially concave contour, such that the device 100 may matingly engage with the user's breast, and/or be placed between a user's breast and bra, and then activated. In this manner, the device 100 may be kept in place without user intervention or additional supporting structures.

FIG. 5 illustrates a top view of one embodiment of devices and a charging cable. As shown in FIG. 5, the devices 200, 205, may each have power connectors 225, 230. A charging cable 210 may connect to the devices 200, 205 by connecting to the power connectors 225, 230. The charging cable 210 may comprise a power source connector 215. The power source connector 215 may comprise a usb connector, or other connector configured to transfer power to the devices 200, 205.

FIG. 6 illustrates a block diagram of the components of the device. As shown in FIG. 6, the device 600 may comprise a micro-controller unit (MCU) 605, power switch 610, heater switch 615, LED 620, heating safety component 625, power port 630, power source 635, motor MOSFET 640, motor 645, heater MOSFET 650, and heater 655. MOSFET stands for metal-oxide-semiconductor field-effect transistor. Other types of heating components and vibrating components may be used, but MOSFETs are currently the most efficient and economical.

The MCU 605 may act as the brain of the device 100, wherein commands and signals may be sent to the MCU 605, such as from the power switch 610, heater switch 615, and heating safety component 625. Based on the signals sent to the MCU 605, the MCU 605 may send signals to control the LED 620, motor MOSFET 640, and heater MOSFET 650. The motor MOSFET 640 and heater MOSFET 650 may then send signals to the motor 645 and heater 655, respectively.

The power switch 610 may be configured to set the vibration motor 645 to one or more vibration modes. In one embodiment, the vibration modes may comprise a constant vibration, pulse vibration, variable vibration, random pulse vibration, or no vibration. In one embodiment, the power switch 610 may comprise a button that selects the vibration mode depending on how many times the power switch 610 is pushed.

The heater switch 615 may be configured to set the heater 655 to one or more heating modes. In one embodiment, the heating modes may comprise low heat, high heat, or no heat. In one embodiment, when the heating mode is set to low heat, the heating safety component 625 may send a signal to cause the heater 655 to stop heating when the heating safety component 625 determines that the temperature has reached or exceeded 45 degrees Celsius. In one embodiment, the heater 655 continues to provide heat for approximately three (3) minutes and then stops heating until the temperature drops to about 42 degrees Celsius. After the temperature reaches 42 degrees Celsius, the heat may be increased until reaching 48 degrees Celsius, and this cycle may repeat until the user deactivates the device 600.

In one embodiment, when the heating mode is set to high heat, the heating safety component 625 may send a signal to cause the heater 655 to stop heating when the heating safety component 625 determines that the temperature has reached or exceeded 48 degrees Celsius. In one embodiment, the heater 655 continues to provide heat for approximately three (3) minutes and then stops heating until the temperature drops to about 45 degrees Celsius. After the temperature reaches 45 degrees Celsius, the heat may be increased until reaching 48 degrees Celsius, and this cycle may repeat until the user deactivates the device 600.

In one embodiment, the heater 655 provides a continuous heat such that the temperature of the device 600 is a substantially constant level at a predetermined temperature.

In one embodiment, the heating safety component 625 may send a signal to cause the heater 655 to stop heating when the heating safety component 625 determines that the temperature has reached or exceeded 52 degrees Celsius, and resume heating when the temperature 35 degrees Celsius.

The device 600 may also be configured to turn off one, two, or both components after being on for thirty (30) minutes.

In one embodiment, the heating safety component 625 may comprise a mechanism for sensing temperature of the device and automatically send a signal to cause the heating component to turn off

The combination of heat and vibration applied simultaneously to the breast may increase milk flow during breastfeeding, and increase overall comfort.

Unless otherwise stated, all measurements, values, ratings, positions, magnitudes, sizes, locations, and other specifications that are set forth in this specification, including in the claims that follow, are approximate, not exact. They are intended to have a reasonable range that is consistent with the functions to which they relate and with what is customary in the art to which they pertain.

The foregoing description of the preferred embodiment has been presented for the purposes of illustration and description. While multiple embodiments are disclosed, still other embodiments will become apparent to those skilled in the art from the above detailed description. The disclosed embodiments capable of modifications in various obvious aspects, all without departing from the spirit and scope of the protection. Accordingly, the detailed description is to be regarded as illustrative in nature and not restrictive. Also, although not explicitly recited, one or more embodiments may be practiced in combination or conjunction with one another. Furthermore, the reference or non-reference to a particular embodiment shall not be interpreted to limit the scope. It is intended that the scope or protection not be limited by this detailed description, but by the claims and the equivalents to the claims that are appended hereto.

Except as stated immediately above, nothing that has been stated or illustrated is intended or should be interpreted to cause a dedication of any component, step, feature, object, benefit, advantage, or equivalent, to the public, regardless of whether it is or is not recited in the claims.

What is claimed is:

1. A device for stimulating milk flow, comprising:
a housing;
a vibrating component;
a heating component;
a power source; and
one or more controllers;
wherein said housing houses said vibrating component and said heating component; and
wherein said one or more controllers are configured to control said vibrating component and said heating component, such that both vibration and heat are provided to a lactating user's breast simultaneously, which stimulates milk flow.

2. The device for stimulating milk flow of claim 1, further comprising a heating safety component;
wherein said heating safety component comprises a thermostat.

3. The device for stimulating milk flow of claim 2, wherein said heating safety component is configured to deactivate said heating component upon detection of a pre-determined temperature level; and
wherein said pre-determined temperature level is a temperature level lower than a temperature level that would cause injury to said user of said device for stimulating milk flow after prolonged contact with said user's skin.

4. The device for stimulating milk flow of claim 3, wherein said injury is at least a third-degree burn.

5. The device for stimulating milk flow of claim 3 wherein said heating safety component causes said heating component to output varying levels of heat, such that the temperature generated increases and decreases in a pre-determined manner in a single use session.

6. The device for stimulating milk flow of claim 1, wherein at least one of said one or more controllers is a heat selection switch.

7. The device for stimulating milk flow of claim 6, wherein said heat selection switch allows selection of at least high heat, low heat, or no heat.

8. The device for stimulating milk flow of claim 1, wherein a bottom portion of said housing comprises an engagement surface.

9. The device for stimulating milk flow of claim 8, wherein said engagement surface substantially comprises silicone.

10. The device for stimulating milk flow of claim 8, wherein said engagement surface is contoured.

11. The device for stimulating milk flow of claim 8, wherein said engagement surface is concave.

12. The device for stimulating milk flow of claim 1, wherein a top portion of said housing comprises said one or more controllers.

13. The device for stimulating milk flow of claim 1, wherein said at least one of said one or more controllers is a heating button.

14. The device for stimulating milk flow of claim 13, wherein said heating button is located on a top portion of said housing.

15. The device for stimulating milk flow of claim 1, further comprising a charging port.

16. A system for stimulating milk flow, comprising:
two vibrating and heating devices;
wherein said each of said two vibrating and heating devices comprises a housing, a vibrating component, a heating component, a heating safety component, an engagement surface, one or more controllers, a power source, and a charging port;
wherein said each of said two housings contains one of said two vibrating components and one of said two heating components; and
wherein each of said one or more controllers are configured to control said vibrating component and said heating component, such that both vibration and heat are provided to a lactating user's breast simultaneously in each of said two vibrating and heating devices.

17. The device for stimulating milk flow of claim 16, wherein said two vibrating and heating devices are physically connected to one another when charging.

18. A device for stimulating milk flow, comprising:
a housing;
a vibrating component;
a heating component;
a power source;
a charging port;
a heating safety component; and
one or more controllers;
wherein said housing houses said vibrating component and said heating component;
wherein said one or more controllers are configured to control said vibrating component and said heating component, such that both vibration and heat are provided to a lactating user's breast simultaneously, which stimulates milk flow;
wherein said heating safety component comprises a thermostat;
wherein said heating safety component is configured to deactivate said heating component upon detection of a pre-determined temperature level;
wherein said pre-determined temperature level is a temperature level lower than a temperature level that would cause injury to said user of said device for stimulating milk flow after prolonged contact with said user's skin;
wherein said injury is at least a third-degree burn;
wherein said heating safety component causes said heating component to output varying levels of heat, such that the temperature generated increases and decreases in a pre-determined manner in a single use session;
wherein at least one of said one or more controllers is a heat selection switch;
wherein said heat selection switch allows selection of at least high heat, low heat, or no heat;
wherein a bottom portion of said housing comprises an engagement surface;
wherein said engagement surface substantially comprises silicone;
wherein said engagement surface is contoured;
wherein said engagement surface is concave;
wherein a top portion of said housing comprises said one or more controllers;
wherein said at least one of said one or more controllers is a heating button; and
wherein said heating button is located on a top portion of said housing.
